(19) United States
(12) Patent Application Publication
(10) Pub. No.: US 2021/0121405 A1
(43) Pub. Date: Apr. 29, 2021

(54) Methods for Formulating an API, Composite Materials, and Solid Unit Dosage Forms

(71) Applicant: KelSie Biotech, LLC, Boulder, CO (US)

(72) Inventors: Lia Rebits, Lakewood, CO (US); Xuno Gildelamadrid, Fort Morgan, CO (US)

(21) Appl. No.: 17/081,512

(22) Filed: Oct. 27, 2020

Related U.S. Application Data
(60) Provisional application No. 62/927,404, filed on Oct. 29, 2019.

(51) Int. Cl. A61K 9/14; A61K 9/20; A61K 9/16

(52) U.S. Cl. CPC A61K 9/146; A61K 45/06; A61K 9/1682; A61K 9/2077

(57) ABSTRACT

A method for formulating an active pharmaceutical ingredient (API) comprises mixing the API and micronized polymer matrix particles comprising polymer strands and having a mean geometric particle diameter in a range of from about 0.1 to about 100 microns, wherein the API is in a liquid phase, to form a homogeneous slurry, allowing the polymer matrix particles to absorb the API, and drying the slurry to form a composite material. The composite material facilitates formation of a tablet or other solid unit dosage form containing the API and/or improves dissolution of the API, for example, in vivo.

### FIELD OF THE INVENTION

The present invention is directed to a method of formulating an active pharmaceutical ingredient (API), which employs micronized polymer matrix particles to form a composite material. The composite material facilitates formation of a tablet or other solid unit dosage form containing the API and/or improves dissolution of the API, for example, in vivo.

### BACKGROUND OF THE INVENTION

Dry powder preparations are commonly used throughout the pharmaceutical, nutraceutical, biotechnological and food industries. Generally, a desirable trait among these types of preparations is the ability of the powder to contribute favorably to the bioavailability of an active ingredient of interest, for example, an active pharmaceutical ingredient (API), by increasing the amount that can be absorbed by the body as compared to a bulk quantity of the ingredient. Increases in bioavailability may lead to greater efficiency, requiring smaller quantities of such an active ingredient to produce a desired effect, beneficially reducing active ingredient waste and potentially resulting in lower production costs. Alternatively, greater bioavailability may also lead to a faster onset of effect, which would be beneficial in many areas of healthcare such as pain management, mental health treatment, and the like.

There are several methods for increasing bioavailability currently employed in practice as well as described in the literature. It is well-known that dissolution of an API needs to occur before absorption into the body can be accomplished, and assays that measure dissolution rate are often used as a surrogate for predicting increases in bioavailability. However, a majority of APIs are hydrophobic and therefore dissolve poorly and slowly in aqueous solutions. A common method for increasing the dissolution kinetics, and thus the bioavailability, of APIs is to create a large surface area, thereby increasing the contact area between an aqueous solution and an API and increasing the rate at which the API will be solvated. This is often accomplished through the creation of small particles of API, on the order of microns to nanometers. Alternatively, membrane permeation enhancer ingredients can be added to an API-containing composition in various forms, for example, as cell-junction modifiers or emulsifying surfactants. These additional ingredients contribute to bioavailability increases by affecting transport across biological membranes, and/or by stabilization of suspended active particles in solution.

Drawbacks of the current technologies for bioavailability enhancement are numerous. Cell-junction modification and surfactant-stabilized suspensions require the introduction of additional ingredients into the preparation that often have not been well-tested for safety, or chemical compatibility with the API. Effectively, these types of preparations include two or more “active” ingredients; the API required to produce the intended therapeutic effect, and other ingredients that facilitate transport across biological membranes, for example by increasing the permeability of such membranes. This increases the complexities of processing, stability, and toxicity profile assessment of such compositions.

Simply reducing the physical size of API particles in an attempt to increase bioavailability also poses challenges. Although dividing the API dosage form into fine particles increases the surface area and thus accelerates the dissolution rate, it also typically creates powder handling issues. Powders composed of small particles of pure API usually present with poor compressibility, which makes solid unit dosage formation, for example in the form of tablets or wafers, difficult, and poor dispersibility, which results in static cling and/or low flowability. Additionally, the increased surface area increases the susceptibility of the powder to moisture uptake from the environment, especially in the case of hygroscopic APIs, and increases the rate of oxidative degradation as compared to larger particles of API. Interaction with taste receptors on the tongue may also be increased, as the API is more easily spread and coated around the mouth, leading to potential patient-compliance issues, as many hydrophobic APIs are quite bitter in taste.

Accordingly, a need remains to provide improved formulation methods for hydrophobic APIs.

### SUMMARY OF INVENTION

It is therefore an object of the invention to provide improved methods for formulating an API. In certain embodiments, the methods provide API formulations which exhibit increased dissolution rates of the API in an aqueous medium, for example as compared with pure API and/or prior art formulation techniques. In certain embodiments, the methods provide API formulations with improved dissolution rates of the API in an aqueous medium while retaining or also providing favorable powder handling characteristics for handling, storage and formulation. In certain embodiments, the methods provide improved handling characteristics for low-melting point APIs which are typically sticky at room temperature or solid unit dosage forming temperatures. In certain embodiments, the methods provide API formulations which exhibit improved compaction properties which allow improved formation of solid unit dosage forms such as tablets and wafers.

In one embodiment, the invention is directed to methods for formulating an active pharmaceutical ingredient (API). The methods comprise mixing the API and micronized polymer matrix particles comprising polymer strands and having a mean geometric particle diameter in a range of from about 0.1 to about 100 microns, wherein the API is in a liquid phase, to form a homogeneous slurry, allowing the polymer matrix particles to absorb the API, and drying the slurry to form a composite material.

In another embodiment, the invention is directed to composite materials which comprise an active pharmaceutical ingredient (API) absorbed on micronized polymer matrix particles comprising polymer stands and having a mean geometer particle diameter in a range of from about 0.1 to about 100 microns.

The methods according to the invention are advantageous in formulating a composite material that in various embodiments exhibits increased dissolution rates of the API in an aqueous medium, for example as compared with pure API and/or prior art formulation techniques, often while retaining favorable powder handling characteristics, provide improved handling characteristics for low-melting point APIs which are typically sticky at room temperature or solid unit dosage forming temperatures, and/or exhibit improved compaction properties which allow improved formation of solid unit dosage forms such as tablets and wafers.

These and additional objects and advantages of the invention will be more evident in view of the following detailed description.

### BRIEF DESCRIPTION OF DRAWINGS

The detailed description of the invention will be more fully understood in view of the drawings, in which:

FIG. 1A shows a scanning electron micrograph (SEM) of micronized microcrystalline cellulose (MCC) particles comprising polymer strands and having ˜5 μm mean geometric particle diameter.

FIG. 1B shows an SEM of a composite material according to the invention prepared as described in the Example.

FIG. 2A shows an SEM of mannitol having ˜50 μm average particle diameter.

FIG. 2B shows an SEM of a comparative mannitol-THC composite material (agglomerated particles) as described in the Example.

The drawings are not to be construed as limiting of the invention in any manner.

### DETAILED DESCRIPTION

The present invention provides improved methods for formulating an API.

Bioavailability of an API which is administered orally, for example, by sublingual or pulmonary administration, or nasally is dependent, in part, on the ability of the API to cross the mucosal membrane. Accordingly, one way to increase bioavailability is to increase the ability of the API to cross the mucosal membrane, for example by transforming the API into a form that exhibits an increased ability to cross the mucosal membrane. As noted previously, several methods of accomplishing this exist, including dividing the API dose into small particles to increase the surface area and thus the dissolution rate, emulsifying the API through the use of surfactants, and/or adding excipients to alter the nature of the mucosal membrane to increase its permeability. The latter options are disadvantageous in that they necessitate the inclusion of potentially undesirable additional ingredients into the formulation. Such additional ingredients may be unsafe for chronic consumption, may be allergens or irritants, and/or may interact chemically with the API, reducing its processing ability and/or shelf stability.

The methods of the present invention increase the surface area of the API and therefore allow for a greater proportion of the API molecules to immediately interact with a solvent upon administration or other contact with an aqueous medium. This accelerates the dissolution rate of the API, and its thermodynamic solubility limit is reached more quickly than for a comparable dose of particles of larger diameter. After a molecule of API is solvated, it has the ability to diffuse through the mucosal membrane, and then passively diffuse through the cell lining, into the bloodstream. If the API particles are slowly solvated, and/or larger clumps of molecules remain for periods of time, passive diffusion through the cell membrane is hindered, and the clump of API will likely be transferred through the gastrointestinal tract before complete dissolution and absorption can take place. Therefore, increasing the proportion of molecules directly in contact with the solvent will accelerate the absorption of the API into the bloodstream, through enhanced dissolution kinetics.

Traditional division of an API dose into fine particles does, however, present drawbacks for the handling characteristics of those particles. The increased surface area of most fine powders increases the Van der Waals and static interactions between neighboring particles, resulting in poor flowability. The void space between many types of API particles also results in very poor compressibility of a powder composed of small particles, leading to greater tablet press equipment strain, and sticking, picking, and capping of tablets due to incomplete cohesion of the granulate.

In order to overcome the issues encountered by finely dividing most APIs into small particles, the methods of the present invention comprise absorbing the API into micronized particles of a polymer matrix that possesses chemical and physical properties which improve handling characteristics of an API. Certain polymers, because of their inherent chemical compositions, retain good handling characteristics even when micronized into small particles. In one embodiment, the polymer matrix particles comprise a hydrophilic polymer. Microcrystalline cellulose (MCC), for example, is a hydrophilic polymer and exhibits good handling characteristics in that it is free flowing and has little or no static cling. MCC is inherently highly compressible, and absorbs over-compression force efficiently in tablet press equipment. Additionally, MCC is very cohesive, and can be tableted without the presence of a lubricating component in the formulation mixture, displaying very little tendency to stick to the tablet press punches. When combined with an API according to the present methods, the MCC particles can retain their advantageous properties, forming a composite particle comprising API and polymer matrix.

The methods and composite materials of the invention are suitable for incorporating any API into a solid formulation. In a specific embodiment, the API is hydrophobic, with a low thermodynamic water solubility limit, and/or slow dissolution kinetics in aqueous media, as the advantages provided by the present invention are particularly useful in formulating hydrophobic APIs. In a specific embodiment, the hydrophobic API has a low melting point, below 80° C., and therefore is, by itself, sticky when formulated into a solid unit dosage form such as by compression. In another specific embodiment, the hydrophobic, low-melting point API is a cannabinoid or cannabinoid acid. Examples include, but are not limited to, cannabidiol (CBD), Δ9-tetrahydrocannabinol (THC), tetrahydrocannabinolic acid (THCA), cannabidiolic acid (CBDA), cannabinol (CBN), cannabigerol (CBG), cannabichromene (CBC), cannabitriol (CBT), or combinations of two or more thereof.

Polymers suitable for use in the invention are suitably hydrophilic and include MCC, methyl cellulose, polyvinyl acetate, polyvinylpyrrolidone, crospovidone, ethyl cellulose, carboxymethyl cellulose, hydroxypropyl methyl cellulose, chitosan, pectinic acid, lactide-co-glycolide polymers, starch, sodium starch glycolate, polyvinyl alcohol, psyllium, gum arabic, guar gum, xanthan gum, and gelatin, and combinations of two or more thereof.

In addition to the chemical nature of the polymer and the advantageous properties it imparts, the intricate matrix of polymer strands in each micronized polymer particle also affords the opportunity to absorb the API into the polymer matrix, spreading out the API dosage and increasing its surface area. By absorbing the API into the polymer matrix of the particles, its flowability and compressibility are increased. Additionally, for low melting point APIs, the stickiness of a low-melting point API is reduced in the composite material as the API is largely within the particles. After immersion into an aqueous medium, the hydrophilic polymer strands will attract water, immersing their absorbed, spread-out API content and facilitating dissolution of the API into the aqueous medium. The pre-micronized nature of the polymer particles also provides a relatively large overall surface area of the composite material and hastens wetting of the API.

The micronized polymer matrix particles comprise pre-formed polymer particles of a defined mean geometric particle diameter. The particles comprising polymer strands are typically non-spherical, but spherical particles comprising polymer strands are also suitable for use in the present invention. The micronized polymer matrix particles have a mean geometric particle diameter of from about 0.1 to 100 microns, measured by laser diffraction. As those of ordinary skill in the art appreciate, measuring a sample by laser diffraction provides an average of all the dimensions of the particles therein. In a more specific embodiment, the micronized polymer matrix particles have a mean geometric particle diameter of from about 1 micron to about 50 microns. In yet more specific embodiments, the micronized polymer matrix particles have a mean geometric particle diameter of from about 1 micron to about 10 microns, or from about 5 microns to about 10 microns. The micronized polymer particles are formed of polymer strands, which allow absorption of the API within the particles and are therefore distinguishable from solid particles. In other embodiments of the invention, the polymer particles are porous but have a morphology other than strands, and the pores allow absorption of the API in a manner as described above in the polymer matrix particles comprising strands.

The API may be absorbed into the polymer matrix particles from a liquid phase in any one of a number of ways, depending on the nature of the API and polymer particles. For low-melting point APIs and high-melting point polymers, a form of melt mixing may be employed in which the mixture is heated to a temperature between that of the two melting points. The molten API can be absorbed into the polymer particles, in experimentally-determined ratios, by means of simple mixing, and then gradually cooled to produce a homogenous composite. Alternatively, a form of wet absorption may be used, in which the particular API is suspended or dissolved in a liquid solvent in which the polymer particles are insoluble. The API suspension or solution and the polymer particles are mixed, in experimentally-determined ratios, into a homogenous slurry, and then gradually dried, for example under heat and/or vacuum, until drying and/or removal of liquid solvent is complete. Blending, milling, and/or grinding of the composite material may optionally be employed, if necessary, to eliminate agglomerates.

The variation in ratio of API and polymer matrix particles may encompass any suitable ratio sufficient for complete homogenization, i.e., absorption of the API in the particles. In specific embodiments, the API and the polymer matrix particles are mixed in a weight ratio of from about 1:1 to about 1:100, or from about 1:1 to about 1:50, or from about 1:1 to about 1:10.

Accordingly, the composite material thus formed will possess bulk powder handling properties that are superior to those of the pure API particles, particularly micronized API particles, especially in the case of low-melting point APIs. In certain embodiments, the composite material exhibits increased dissolution of the API, for example, as compared with pure API particles, and/or increased bioavailability of the API after immersion in an aqueous liquid, due to the increased area of interaction of the API with the aqueous liquid within the polymer matrix particles. Increased bioavailability is typically assessed by assay dissolution rate studies. The invention thus solves a common industry problem by increasing API dissolution rate while simultaneously retaining or providing favorable powder-handling characteristics.

In a specific embodiment in which the API has a low-melting point, for example less than 80° C., the low-melting point API is absorbed into the micronized polymer matrix particles and the resultant composite exhibits an increase in desirable handling characteristics, for example, defined by a reduction of stickiness, cohesion, aggregation, hygroscopicity, and/or deformation of the API-containing particles.

In yet further embodiments of the invention, the composite material of the invention is included in a solid unit dosage form. Such forms include, but are not limited to tablets, wafers, capsules, and the like. The composite material is especially suitable for use in solid unit dosage forms produced using compaction, for example in formation of tablets and wafers. The composite material is advantageous for use in such dosage forms in that it exhibits increased flowability and better processibility, and compositions including the composite material exhibit reduced sticking, picking and capping with compaction equipment such as tablet press tooling. This also reduces equipment stress through the retention of good compressibility characteristics imparted by the flexible composite material including the polymer matrix particles.

In a specific embodiment, a tablet or wafer comprises the composite material according to the invention; sugar, sugar alcohol, or a combination thereof; silica, silicified microcrystalline cellulose, or a combination thereof; and lubricant comprising sodium stearyl fumarate and lecithin. In a more specific embodiment, a tablet or wafer comprises from about 1 to about 25 wt %, more specifically from about 5 to about 20 wt %, of the composite material according to the invention; from about 45 to about 80 wt %, more specifically from about 55 to about 75 wt %, sugar, sugar alcohol, or a combination thereof; from about 1 to about 20 wt %, more specifically from about 1 to about 15 wt %, silica, silicified microcrystalline cellulose, or a combination thereof; and from about 0.5 to about 5 wt % lubricant comprising sodium stearyl fumarate and lecithin. In more specific embodiments, suitable sugars and sugar alcohols include, but are not limited to, fructose, galactose, glucose, lactose, maltose, sucrose, arabinose, dextrose, fucose, mannose, ribose, rhamnose, trehalose, xylose, mannitol, sorbitol, xylitol, isomalt, arabitol, ribitol, galactitol, pharmaceutically acceptable inositol such as myo-inositol, maltitol, lactitol, iditol, and fucitol, or, more specifically, the tablet or wafer includes mannitol. In more specific embodiments, the tablet or wafer comprises a combination of sodium stearyl fumarate and lecithin in an amount of from about 1 to about 4 wt %, and at least 50 wt % sodium stearyl fumarate, based on the combined weight of sodium stearyl fumarate and lecithin. In yet more specific embodiments of the tablet or wafer, the API is a cannabinoid as described above, or a mixture of such cannabinoids.

The tablets or wafers may be provided with one or more additional conventional excipients, as long as such conventional excipients. In a specific embodiment, such additional conventional excipient does not interfere with the ability of the tablet or wafer compositions to be efficiently formed into a solid dosage unit by direct compression. Examples include crospovidone, which acts as a disintegrant, flavoring agents, coloring agents, preservatives, other benefit-providing additives such as melatonin, caffeine, GABA (gamma-aminobutyric acid) or other amino acids, and the like. Similarly, the tablets or wafers may be provided with one or more coatings as desired subsequent to the tableting procedure. In a specific embodiment, the tablets include a disintegrant such as crospovidone and are quickly dissolvable upon oral administration. For example, the disintegrant is included in an amount sufficient to dissolve the tablet in a period of from about 10 seconds to about 3 minutes when administered by mouth, for example, as a sublingual tablet. In a specific embodiment, the tablets include from about 1 to about 20 wt %, more specifically, from about 1 to about 10 wt %, of a disintegrant, and in further embodiments, the disintegrant is crospovidone.

Various aspects of the methods and composite materials of the invention are illustrated in the following Example.

### Example

A composite material according to the invention was prepared. Specifically, tetrahydrocannabinol (THC) (100 g) was melted by heating in a glass jar to ˜50° C. in a water bath for 10 minutes until the viscosity of the compound was reduced to a syrupy consistency. The contents of the jar were poured onto a 1′×2′ tray covered with wax paper, and the tray was rotated so that the molten THC was thinly distributed over the paper. The tray was placed in a freezer (−20° C.) for several hours, until the THC was congealed into a glass-like consistency. The tray was removed from the freezer and the glassified THC was quickly removed from the paper, broken into small pieces, and distributed evenly on a bed of micronized microcrystalline cellulose (MCC) particles (400 g), comprising stranded polymer and having a mean geometric diameter of about 5 microns, which had been previously placed into the ceramic insert of a slow cooker. The cooker was heated to 50-60° C. until the frozen THC pieces were returned to a molten, low-viscosity consistency. The THC-MCC mixture was stirred continuously until the THC was incorporated throughout the MCC bed. An immersion blender was used to break up areas of unincorporated THC and homogenize the mixture. The mixture was allowed to cool for about an hour to room temperature, and was placed into a blender chamber. The mixture was blended in a pulsing manner for 15 minutes; periodically, pauses were taken to remove any accumulated material from the sides of the blending container.

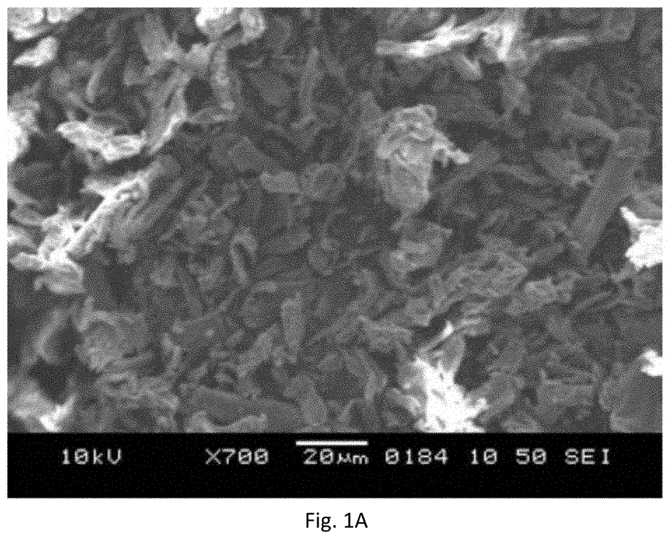

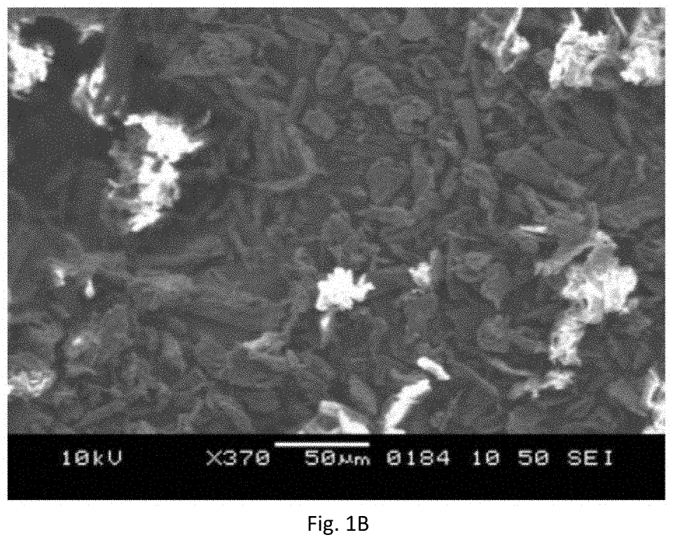

The micronized MCC polymer matrix particles are shown as-received in the scanning electron micrograph (SEM) in FIG. 1A, while the composite material, prepared as described in the preceding paragraph, is shown in the SEM of FIG. 1B. As can be seen from the figures, the approximate size and overall appearance of the polymer particles remains unchanged, indicating that the THC has been absorbed into the matrix particles, with no observable change in the polymer particles as seen by SEM.

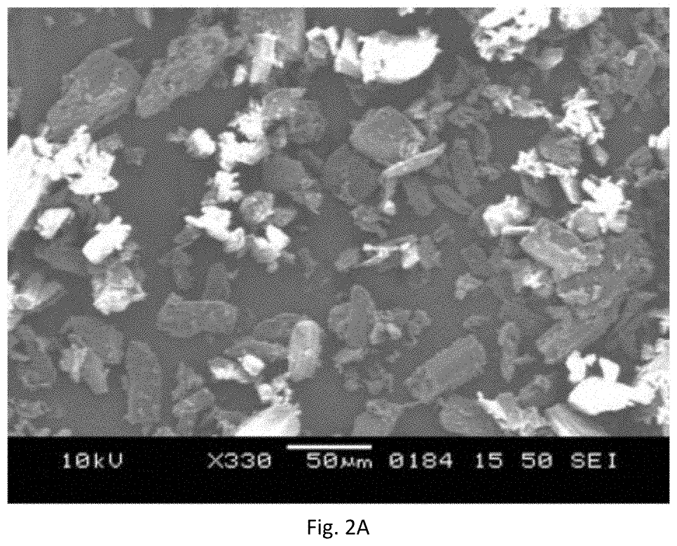

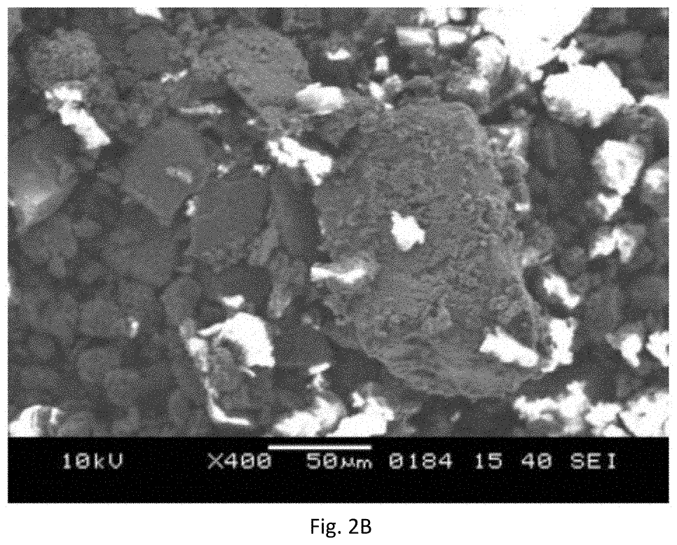

As an experimental control, a composite material was prepared using a method as described above except that mannitol particles having a mean particle diameter of about 50 microns was used instead of the micronized MCC. The as-received mannitol is shown in the SEM of FIG. 2A, and the THC-mannitol composite particles are shown in FIG. 2B. As can be observed, the composite particles are more agglomerated than the as-received mannitol particles. It can be speculated that the non-polymer, mannitol matrix particles did not absorb the THC. The composite material particles were sticky due to the inherent stickiness of the THC, and agglomeration resulted.

The specific embodiments and examples described in the present disclosure are illustrative only in nature and are not limiting of the invention defined by the following claims. Further aspects, embodiments and advantages of the methods and composite materials of the present invention will be apparent in view of the present disclosure and are encompassed within the following claims.

What is claimed is:

1. A method for formulating an active pharmaceutical ingredient (API), comprising mixing the API and micronized polymer matrix particles comprising polymer strands and having a mean geometric particle diameter in a range of from about 0.1 to about 100 microns and, wherein the API is in a liquid phase, to form a homogeneous slurry, allowing the polymer matrix particles to absorb the API, and drying the slurry to form a composite material.

2. The method of claim 1, wherein the micronized polymer matrix particles have a mean geometric particle diameter in a range of from about 1 to about 50 microns, from about 1 to about 10 microns, or from about 5 to about 10 microns.

3. The method of claim 1, wherein the micronized polymer matrix particles comprise a hydrophilic polymer.

4. The method of claim 1, wherein the micronized polymer matrix particles comprise microcrystalline cellulose, methyl cellulose, polyvinyl acetate, polyvinylpyrrolidone, crospovidone, ethyl cellulose, carboxymethyl cellulose, hydroxypropyl methyl cellulose, chitosan, pectinic acid, lactide-co-glycolide polymers, starch, sodium starch glycolate, polyvinyl alcohol, psyllium, gum arabic, guar gum, xanthan gum, gelatin, or a combination of two or more thereof.

5. The method of claim 1, wherein the API has a melting point less than about 80° C.

6. The method claim 1, wherein the API comprises a cannabinoid or cannabinoid acid.

7. The method of claim 1, wherein the API has a melting point lower than a melting point of the micronized polymer matrix particles and the API is provided in a liquid phase by melting the API at a temperature lower than the melting point of the micronized polymer matrix particles.

8. The method of claim 7, wherein the composite material is dried by cooling the composite material to a temperature below the melting point of the API.

9. The method of claim 1, wherein the API is provided in liquid form by dissolving or suspending the API in a liquid in which the micronized polymer matrix particles retain their particle form.

10. The method of claim 9, wherein the composite material is dried by removing the liquid in which the API is dissolved or suspended.

11. The method of claim 1, further comprising blending the composite material to deagglomerate any agglomerated particles.

12. The method of claim 1, wherein the API and the micronized polymer matrix particles are mixed in a weight ratio of from about 1:1 to about 1:100, or from about 1:1 to about 1:50, or from about 1:1 to about 1:10.

13. A composite material comprising an active pharmaceutical ingredient (API) absorbed on micronized polymer matrix particles comprising polymer strands and having a mean geometric particle diameter in a range of from about 0.1 to about 100 microns, from about 1 to about 50 microns, from about 1 to about 10 microns, or from about 5 to about 10 microns.

14. The composite material of claim 13, wherein the micronized polymer matrix comprises a hydrophilic polymer.

15. The composite material of claim 13, wherein the micronized polymer matrix particles comprise microcrystalline cellulose, methyl cellulose, polyvinyl acetate, polyvinylpyrrolidone, crospovidone, ethyl cellulose, carboxymethyl cellulose, hydroxypropyl methyl cellulose, chitosan, pectinic acid, lactide-co-glycolide polymers, starch, sodium starch glycolate, polyvinyl alcohol, psyllium, gum arabic, guar gum, xanthan gum, gelatin, or a combination of two or more thereof.

16. The composite material of claim 13, wherein the API has a melting point less than about 80° C.

17. The composite material of claim 13, wherein the API comprises a cannabinoid or cannabinoid acid.

18. The composite material of claim 13, comprising the API and the micronized polymer matrix particles in a weight ratio of from about 1:1 to about 1:100, or from about 1:1 to about 1:50, or from about 1:1 to about 1:10.

19. A solid unit dosage form comprising the composite material of claim 13.

20. The unit dosage of claim 19 in the form of a tablet or wafer.

21. A method for formulating an active pharmaceutical ingredient (API), comprising mixing the API and micronized polymer matrix particles comprising pores and having a mean geometric particle diameter in a range of from about 0.1 to about 100 microns and, wherein the API is in a liquid phase, to form a homogeneous slurry, allowing the polymer matrix particles to absorb the API, and drying the slurry to form a composite material.
